(19) United States
(12) Patent Application Publication
(10) Pub. No.: US 2010/0285127 A1
(43) Pub. Date: Nov. 11, 2010

(54) TASTE MASKED PHARMACEUTICAL COMPOSITIONS OF S-ALKYLISOTHIOURONIUM DERIVATIVES

(76) Inventors: Refael Barkan, Rishon Le Zion (IL); David Coffin-Beach, St. Augustine, FL (US); Victor Ghicavii, Chisinau (MD)

Correspondence Address: FENNEMORE CRAIG, 3003 NORTH CENTRAL AVENUE, SUITE 2600, PHOENIX, AZ, 85012, US

(21) Appl. No.: 12/663,804

(22) PCT Filed: Jun. 13, 2007
(86) PCT No.: PCT/IL07/00714

§ 371 Date: Jul. 19, 2010

(51) Int. Cl. A61K 9/36; A61P 25/06; A61P 1/08; A61K 31/661

(52) U.S. Cl. 424/480; 514/76

(57) ABSTRACT

The present invention relates to taste masked compositions of S-alkylisothiouronium derivatives, including, but not limited to, S-ethylisothiouronium diethylphosphate, in the form of a coated oral tablet having a desirable dissolution profile.

### FIELD OF THE INVENTION

The present invention relates to compositions of S-alkylisothiouronium derivatives, including, but not limited to, S-ethylisothiouronium diethylphosphate, in the form of a coated oral tablet having a pleasant taste and desirable dissolution profile.

### BACKGROUND OF THE INVENTION

### S-alkylisothiouronium Compounds

Various S-alkylisothiouronium salts with phosphorus containing acids, among them Difetur (S-ethylisothiouronium diethylphosphate), have been studied and were shown to have radioprotective effects (Zherebchenko, et al., Radiobiologya, 8:582-587 (1968); Goloschapova, et al. Radiobiology, 21:521-525 (1981)).

International Patent Publication No. WO 98/13036 to the applicant of the present invention discloses the use of S-alkylisothiouronium derivatives, including several novel compounds, as medicaments for increasing arterial blood pressure or for protecting subjects against hyperoxia. The compounds are disclosed for the treatment of acute hypotension, e.g., shock conditions and chronic arterial hypotension or oxygen poisoning. The invention is exemplified by the hypertensive effect of S-ethylisothiouronium diethylphosphate under various conditions.

International Patent Publication No. WO 02/19961 to the applicant of the present invention teaches the use of S-alkylisothiouronium derivatives for treating headache, migraine, or nausea and vomiting. According to that disclosure, S-alkylisothiouronium derivatives can be administered orally in a solid form such as in tablets, pills, dragees, and capsules or in a liquid form.

The S-alkylisothiouronium compounds were shown to be highly efficacious when administered orally in the form of a tablet. Yet, the tablets were reported to be unpalatable. Without wishing to be bound to theory, the bad taste may be due to the hydrolytic action of atmospheric moisture on the tablet formulation and the formation of highly aromatic hydrolysis products of the active ingredient.

Certain compositions useful for masking an unfavorable taste of pharmaceuticals are known in the art.

For example, U.S. Pat. No. 4,916,161 relates to a process for taste masking pharmaceutical agents including ibuprofen by wet-granulating a dry particulate/pre-granulation blend of the agent and hydroxypropyl methylcellulose phthalate (HPMCP) with an aqueous composition in which the HPMCP is at least partly soluble.

U.S. Pat. No. 4,252,786 teaches a film coated controlled release tablet comprising: (i) a compressed matrix comprising an effective amount of medicament dispersed in a blend of 1:10 to 10:1 parts by weight polymeric vinyl pyrrolidone and a carboxyvinyl hydrophilic polymer; and (ii) a water insoluble, water permeable film coating on the compressed matrix, the film coating having a thickness of about 1 to 15 mil. and comprising a blend of hydrophobic and hydrophilic polymers.

International Patent Publication No. WO 02/096392 is directed to a taste-masked formulation which allegedly reduces or eliminates the release of active ingredient in the mouth and yet will rapidly release the active ingredient in acidic conditions, such as those found in the stomach. Those formulations comprise taste-masked particles which include (a) a predetermined amount of a particulate active ingredient; (b) at least one coating layer coating the particulate active ingredient. That application states that modified celluloses such as hydroxypropylmethyl cellulose (“HPMC”), ethylcelluloses and mixtures of celluloses, as a single coating layer are ineffective in taste-masking certain orally disintegrating tablets containing particularly offensive tasting water-soluble active ingredients such as, cetirizine hydrochloride.

The unpleasant taste of S-alkylisothiouronium tablets has been a complaint from subjects in the clinical trials. There remains an unmet need for tablets comprising S-alkylisothiouronium derivatives, which leave no foul taste following oral administration to a subject.

### SUMMARY OF THE INVENTION

The family of S-alkylisothiouronium phosphate derivatives, in particular S-ethylisothiouronium diethylphosphate (Difetur), tend to have a bad smell and unpleasant taste when formulated into tablets. The present invention provides taste masked tablets comprising as an active ingredient at least one S-alkylisothiouronium phosphate derivative coated with at least one layer of a polymeric coating agent. The coated tablet formulation for oral administration is palatable in taste tests and has a desirable dissolution profile. The tablets are useful in the treatment of various diseases and disorders and can be prepared in an assortment of dosages.

Thus, according to one aspect of the present invention there is provided a pharmaceutical composition for oral administration having a masked taste, comprising as an active agent at least one S-alkylisothiouronium derivative; and a taste masking effective amount of at least one polymeric coating.

In some embodiments the polymeric coating comprises a cellulose based compound. In various embodiments the cellulose based coating comprises a hydrophilic, water soluble polymer and includes, but is not limited to, cellulose alkyl ethers such as methyl cellulose (MC), ethyl cellulose, hydroxypropylmethyl cellulose (HPMC), hydroxymethyl cellulose phthalate (HMCP), hydroxypropyl cellulose (HPC), cellulose acetate phthalate, and cellulose acid phthalate.

A currently preferred polymer coating comprises hydroxypropylmethyl cellulose and polyethylene glycol. The film forming material or binder employed in the coating preferably comprises Opadry Clear® which contains hydroxypropyl methylcellulose and polyethylene glycol; and/or Opadry White® which contains hydroxypropyl methylcellulose, polyethylene glycol and titanium dioxide

In some embodiments the coating comprises a solvent based composition, including ethyl cellulose and or cellulose acid phthalate.

The coating may be applied by any conventional technique such as pan coating or spray coating.

The coating can comprise additional agents, including coloring and flavoring agents. The coating preferably comprises a flavoring agent. The flavoring agent can be a sweet flavoring agent (sweetener), which can be combined with a sour flavoring agent, a bitter flavoring agent, or mixtures thereof. The flavoring agent is selected from natural flavors, natural fruit flavors, artificial flavors, artificial fruit flavors, flavor enhancers, and mixtures thereof. These flavoring agents can be used in combination with a sweetener, a sour flavoring agent, a bitter flavoring agent, or mixtures thereof. The sweetener is a natural sugar or a sugar substitute of artificial origin.

In various embodiments the coating comprises more than one film coating layer. In some embodiments the coating layer comprises a precoat and a final coat. In other embodiments the coating layer further comprises a polishing coat.

In some embodiments the S-alkylisothiouronium derivative is a compound having the general formula I:

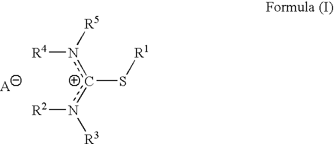

wherein,
R₁ is a linear or branched, saturated or unsaturated alkylene, comprising one to eight carbon atoms, optionally substituted with one or more substituent selected from the group consisting of halogen, primary, secondary or tertiary amine, primary, secondary or tertiary alcohol, or interrupted by one or more heteroatom selected from the group consisting of O, N, and S;
R₂, R₃, R₄ and R₅ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion derived from a phosphorous containing acid.

In various embodiments the anion is selected from the group consisting of an anion derived from a phosphorus acid ester, and an amide. In preferred embodiments the anion is derived from a mono or di-alkyl ester of a phosphorous containing acid.

According to still further features in the described embodiments the compound is selected from the group consisting of:

S-methylisothiouronium methylphosphite;

S-methylisothiouronium dimethylphosphate;

S-ethylisothiouronium metaphosphate;

S-ethylisothiouronium ethylphosphite;

S-ethylisothiouronium diethylphosphate;

S-propylisothiouronium propylphosphite;

S-isopropylisothiouronium metaphosphate;

S-isopropylisothiouronium isopropylphosphite;

S-butylisothiouronium dibutylphosphate; and

S-isobutylisothiouronium isobutylphosphite.

A currently preferred compound is S-ethylisothiouronium diethylphosphate.

According to still further features in the described embodiments each of the tablets includes between 10 and 200 mg of the compound.

According to still further features in the described embodiments each of the tablets includes between 20 and 100 mg of the compound.

According to still further features in the described embodiments each of the tablets includes between 30 and 80 mg of the compound. In various embodiments each taste masked tablet comprises as active agent 50 mg S-ethylisothiouronium diethylphosphate.

According to still further features in the described embodiments, the taste masked composition is packaged and identified as having activity for treating or preventing one or more indications selected from: headache, migraine, nausea, emesis, low arterial blood pressure and hyperoxia.

According to still further features in the described embodiments the therapeutically effective amount is selected such that in less than 60 minutes following administration a substantial relief in symptoms is experienced. In other embodiments a therapeutically effective amount is selected such that in less than 50 minutes, less than 45 minutes, less than 40 minutes, less than 35 minutes and less than 30 minutes, a substantial relief in symptoms is experienced.

According to various embodiments the present invention provides a taste masked composition comprising

about 5% w/w to about 50% w/w of at least one S-alkylisothiouronium derivative according to formula I;

about 20% to about 90% of at least one filler-binder;

about 0.2% w/w to about 10% w/w of at least one lubricant, one glidant or a combination thereof;

about 0.2% to about 10% of at least one disintegrant; and

at least one polymer coating.

All components of the tablet are provided as percent weight per weight of the tablet.

In various embodiments the composition of the present invention comprises

about 5% w/w to about 50% w/w of S-alkyisothiuronium diethylphosphate;

about 20% to about 70% of at least one filler-binder selected from the group consisting of anhydrous lactose, microcrystalline cellulose, and a combination of lactose and microcrystalline cellulose;

about 0.2% w/w to about 10% w/w of at least one lubricant, one glidant or a combination thereof selected from the group consisting of colloidal silicon dioxide, stearic acid, talc, calcium stearate, magnesium stearate and sodium stearyl fumarate;

about 0.2% w/w to about 10% w/w of at least one disintegrant selected from crosslinked polyvinyl pyrrolidone, corn starch, potato starch, maize starch, modified starch, croscarmellose sodium, crospovidone, sodium starch glycolate, and mixtures thereof; and

at least one polymer coating comprising hydroxypropylmethylcellulose and polyethylene glycol.

According to a certain embodiment the taste masked composition of the present invention comprises

about 25% w/w of S-alkyisothiuronium diethylphosphate;

about 70% of at least one filler-binder selected from the group consisting of anhydrous lactose, microcrystalline cellulose, and a combination of lactose and microcrystalline cellulose;

about 0.5% w/w of colloidal silicon dioxide;

about 2% w/w of stearic acid;

about 2% w/w of crospovidone;

a polymer precoat comprising hydroxypropylmethylcellulose and polyethylene glycol; and a polymer coating comprising hydroxypropylmethylcellulose and polyethylene glycol.

A currently preferred flavoring agent is vanillin, which is added to the coating composition.

In another aspect, the present invention further provides a method for the preparation of a taste masked tablet composition comprising as an active agent at least one S-alkylisothiouronium derivative, the method comprising the steps of

a) blending a mixture of about 5% final w/w to about 50% final w/w of at least one S-alkylisothiouronium derivative according to formula I with 10% final w/w to about 40% final w/w filler binder and about 0.2% final w/w to about 10% final w/w of at least one lubricant;

b) adding to said blended mixture about 10% final w/w to about final 50% w/w filler binder, and blending;

c) adding to the blended mixture of step b) about from 0.2% final w/w to about 10% final w/w of at least one disintegrant; and about 0.2% final w/w to about 10% final w/w of at least one lubricant and blending;

d) compressing the composition into tablets;

e) precoating the tablets with a polymer composition comprising hydroxypropylmethylcellulose and polyethylene glycol, and an optional flavoring agent;

f) coating the tablets with a polymer composition comprising hydroxypropylmethylcellulose and polyethylene glycol, and an optional flavoring agent.

In some embodiments the tablet is further coated with a polishing composition, which can be the same or different from the precoating composition. In various embodiments the tablet further comprises a final polishing coat comprising a composition comprising water and PEG 8000. Without wishing to be bound to theory the final polishing coat impart sheen to the tablet.

According to certain preferred embodiments the compound is administered following onset of symptoms of a headache, in particular a migraine, or nausea, low blood pressure or exposure to radiochemicals.

According to additional preferred embodiments the compound is administered upon onset of a headache, particularly a migraine, or nausea; low blood pressure or exposure to radiochemicals.

It is understood that the while migraine is the most severe form of headache, the methods of treatment of the present invention are suitable also for other types of headaches and nausea, including but not limited to PMS or hangover associated headaches and nausea. This is particularly appropriate due to the negligible side effects observed in human subjects with the compositions and methods of the present invention.

Compounds of formula (I) inhibit emesis. The compounds are therefore also of use as anti-emetic agents, i.e. in the prevention and treatment of nausea and vomiting. The compounds are especially valuable for the prevention of emesis induced by cancer chemotherapeutic agents such as cisplatin. Particular mention may also be made of the treatment of radiation-induced emesis. Thus, the compounds of formula (I) may be used in the prevention of emesis induced by radiation therapy, e.g. irradiation of the thorax or abdomen, such as in the treatment of cancer; or in the treatment of radiation sickness. It will be appreciated that the compounds of formula (I) may be used prophylactically and references in this specification to treatment include prophylactic treatment as well as the alleviation of acute symptoms.

According to still further features in the described preferred embodiments the step of administering the compound is effected at or prior to onset of nausea. It will be appreciated by the skilled artisan that oral administration may be less desirable after onset of nausea.

The present invention successfully addresses the shortcomings of the presently known medications by providing an efficient compound for treating and/or alleviating the symptoms of headache, in particular migraine, or nausea. The currently preferred composition had no apparent side effects, was palatable to the tester, was shown to be potent in low doses and to elicit a therapeutic/relieving effect within a short time period as compared to currently marketed drugs.

These and further embodiments will be apparent from the figure, detailed description and examples that follow.

### BRIEF DESCRIPTION OF THE FIGURE

FIG. 1 shows a dissolution profile of the taste masked composition comprising S-alkyisothiuronium diethylphosphate (50 mg/tablet) and a precoating and coating layer comprising HPMC and polyethylene glycol.

### DESCRIPTION OF THE PREFERRED EMBODIMENTS

The present invention relates to a novel formulation of S-alkylisothiouronium derivatives which provides a good dissolution profile and is taste masked. The taste masked composition has been tested and was shown to be palatable and provide effective relief for the treatment of headache, in particular migraine, nausea and vomiting; hyperoxia and low blood pressure.

The compositions of the invention are effective in preventing or alleviating emesis associated with migraine or other medical conditions such as chemotherapy or radiotherapy, as well as other symptoms of migraine including phonophobia and photophobia. These compounds were known before to affect arterial blood pressure in cases of acute hypotension (e.g., following hemorrhage, trauma, shock or poisoning).

Before explaining at least one embodiment of the invention in detail, it is to be understood that the invention is not limited in its application to the details set forth in the following description or exemplified in the Examples section. The invention is capable of other embodiments or of being practiced or carried out in various ways. Also, it is to be understood that the phraseology and terminology employed herein is for the purpose of description and should not be regarded as limiting.

As used herein, the term “migraine” is understood expansively to include a subset of headache characterized by unusual severity, unilateral, throbbing, headache pain persisting for 4-72 hours and can include also one or more of the following symptoms: nausea, vomiting, sensitivity to light and/or sounds with or without a preceding “aura” and visual photophobia (e.g., visual disturbances).

According to one aspect of the present invention there is provided an anti-headache, anti-migraine, anti-nausea or anti-emesis medicament comprising, as an active ingredient, a compound having the general formula (I):

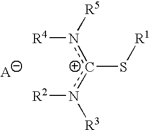

wherein,
R₁ is a linear or branched saturated or unsaturated alkylene, comprising one to eight carbon atoms optionally substituted with one or more substituents selected from the group consisting of halogen, primary or secondary amine, primary or secondary alcohol, or interrupted by one or more heteroatom selected from the group consisting of O, N, and S;
R₂, R₃, R₄ and R₅ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
together with and a pharmaceutically acceptable carrier or diluent.

Preferably, the physiologically acceptable anion is derived, without limitation, from a phosphorus containing acid, the group consisting of an anion derived from a phosphorus containing acid, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, hydrochloride, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

According to currently preferred embodiments of the invention described below, the physiologically acceptable anion is an anion derived from a phosphorus containing acid, more preferably the group consisting of an anion derived from a phosphorus acid ester or amide, most preferably the anion is derived from a mono or di-alkyl ester of a phosphorous containing acid.

As used herein and in the claims, the term “alkylene” refers to a saturated or unsaturated hydrocarbon chain including straight chain or branched chain alkyl, alkenyl or alkynyl.

As used herein, the term “alkyl” refers to a saturated hydrocarbon chain containing 1 to 30, preferably 1 to 6 carbon atoms, such as, but not limited to, methyl, ethyl, n-propyl, isopropyl, n-butyl, isobutyl, tert-butyl, n-pentyl, n-hexyl, and the like. As used herein the term alkyl also reads on haloalkyls, which contain halogen atoms. Alkyl also includes heteroalkyl with heteroatoms of sulfur, oxygen and nitrogen.

“Alkenyl” and “alkynyl” are used to mean straight or branched chain hydrocarbon groups having from 2 to 12 carbons and unsaturated by a double or triple bond respectively, such as vinyl, allyl, propargyl, 1-methylvinyl, but-1-enyl, but-2-enyl, but-2-ynyl, 1 methylbut-2-enyl, pent-1-enyl, pent-3-enyl, 3-methylbut-1-ynyl, 1,1-dimethylallyl, hex-2-enyl and 1-methyl-1-ethylallyl;

The term “cycloalkyl” is used herein to mean cyclic radicals, including but not limited to, cyclopropyl, cyclopentyl, cyclohexyl, and the Ike.

The term “cycloalkylalkyl” as used herein refers to a cycloalkyl group appended to a lower alkyl radical, including, but not limited to cyclohexylmethyl.

The “alkoxyalkyl” mentioned for R substitutes is preferably a group containing a total of 1-22 carbon atoms. As example, methoxyethyl, methoxypropyl, methoxybutyl, ethoxyethyl, ethoxypropyl, ethoxybutyl, n-propoxyethyl, and iso-propoxyethyl, can be mentioned.

The term “alkoxy” as used herein refers to an alkyl group attached to the parent molecular group through an oxygen atom.

The term “alkoxyalkoxy” as used herein refers to an alkoxy group attached to the parent molecular group through an alkoxy group.

The term “halo” or “halogen” as used herein refers to I, Br, Cl or F.

The term “carboxy” as used herein refers to the radical —COOH. The term “ester” refers to —COOR; and the term “amide” refers to —CONH₂ or —CONHR or —CONR₂. The term “cyano” as used herein refers to the radical —CN.

Phosphorus containing and other salts of S-alkylisothiouronium synthesized in a variety of ways, which are well known in the art, for example by alkylating thiourea with appropriate trialkylphosphates or dialkylphosphites while heating in an organic solvent.

Without excluding other options, which are listed below, presently S-ethylisothiouronium diethylphosphate is the preferred compound for the treatment of headache, in particular migraine, and nausea or vomiting. Other examples of S-alkylisothiouronium derivatives which can be used to treat migraine according to the present invention include, but are not limited to, S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutylisothiouronium isobutylphosphite.

These compounds are known to be safe for human use as it is well known in the art that phosphorus containing derivatives of S-alkylisothiouronium have a low toxicity and their LD₅₀ (lethal dose 50%) is in the range of 100-1000 mg/kg, which is far above the therapeutic doses of these compounds, which is the range of about 0.5 to about 5 mg/kg.

### Pharmaceutical Compositions

As used herein a “pharmaceutical composition” refers to a preparation of one or more of the compounds described herein, or physiologically acceptable salts or prodrugs thereof, with other chemical components such as physiologically suitable carriers and excipients, in the form of a taste masked coated table for oral administration.

For oral administration, the compounds can be formulated readily by combining the active compounds with pharmaceutically acceptable carriers well known in the art. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

### Other Pharmaceutical Excipients

Pharmaceutical compositions according to the invention may also comprise one or more filler-binder, lubricating agents, suspending agents, sweeteners, flavoring agents, preservatives, buffers, wetting agents, disintegrants, effervescent agents, and other excipients. Such excipients are known in the art.

Examples of filler-binder agents are lactose monohydrate, lactose anhydrous, and various starches; various celluloses and cross-linked polyvinylpyrrolidone, microcrystalline cellulose, such as Avicel® PH101 and Avicel® PH102, and silicified microcrystalline cellulose (ProSolv SMCC®). A preferable filler binder is a combination of anhydrous lactose and microcrystalline cellulose.

Suitable binding agents including but not limited to lactose, methyl cellulose, hydroxypropyl cellulose, hydroxypropylmethyl cellulose, gelatin, gum arabic, ethyl cellulose, polyvinyl alcohol, pullulan, pregelatinized starch, agar, tragacanth, sodium alginate, propyleneglycol alginate, and the like

Suitable lubricants, including agents that act on the flowability of the powder to be compressed, include but are not limited to colloidal silicon dioxide, such as Aerosil® 200 (fumed silica), talc, stearic acid, magnesium stearate, calcium stearate, polyoxyl stearate, hydrogenated castor oil, dimethylpolysiloxane, microcrystalline wax, yellow beeswax, white beeswax and silica gel.

Suitable disintegrants include lightly crosslinked polyvinyl pyrrolidone, corn starch, potato starch, maize starch, and modified starches, croscarmellose sodium, crospovidone, sodium starch glycolate, and mixtures thereof.

Examples of sweeteners are any natural or artificial sweetener. Reduced-calorie sweeteners include but are not limited to, erythritol, hydrogenated starch hydrosylates, isomalt, lactitol, maltitol, mannitol, sorbitol and xylitol, sucralose, isomalt, aspartame, saccharin, lacitol. Other suitable sweeteners include xylose, ribulose, glucose, mannose, galactose, fructose, sucrose, maltose, corn syrup and corn syrup solids.

Flavoring agents may be a single compound or a blend of compounds, which provide a particular flavor to the tablet. The compounds may be natural or synthetic, solid or liquid, for example an oil or extract. Examples of flavoring agents are monoammonium glycyrrhizinate (Magnasweet®), spice and fruit flavors, and the like. Examples of flavoring agents include mint, fruit, spice and the like. A currently preferred flavoring agent is vanillin.

Preservatives, including anti-oxidant agents, are not required in the formulation but may be optionally added. Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

The anti-oxidant includes dibutylhydroxytoluene (BHT), propyl gallate, butylhydroxyanisol (BHA), α-tocopherol, citric acid, etc.

The surfactant includes polyoxyethylene hardened castor oil, glyceryl monostearate, sorbitan monostearate, sorbitan monopalmitate, sorbitan monolaurate, polyoxyethylene polyoxypropylene block copolymers, polysorbates, sodium laurylsulfate, macrogols, sucrose esters of fatty acids, etc.

The coating agent is selected from a hydrophilic polymer and more preferably a cellulose based polymer, including solvent based celluloses, and includes without limitation hydroxypropylmethyl cellulose, hydroxypropyl cellulose, methyl cellulose, ethyl cellulose, hydroxypropylmethyl cellulose phthalate, hydroxypropylmethyl cellulose acetate succinate, carboxymethylethyl cellulose, cellulose acetate phthalate, cellulose acid phtalate and the like.

The coating agents may further comprise one or more colorants. Suitable colorants include natural and synthetic agents. Additionally, the coating material may comprise a plasticizer. Suitable plasticizers include acetylated monoglyceride, dibutyl tartrate, glycerin, triethyl citrate, triacetin, polyethylene glycol and the like.

The amount of a composition to be administered will, of course, be dependent on the subject being treated, the severity of the affliction, the manner of administration, the judgment of the prescribing physician, etc.

Each of the tablets of the present invention preferably contains between 10 and 300 mg, preferably 20 and 200 mg, more preferably between 30 and 80 mg of the active compound (S-alkylisothiouronium derivatives). As used herein the term “about” refers to ±20%.

As used herein the term “therapeutically effective amount” or “therapeutically efficient” as to a drug dosage, refer to dosage that provides the specific pharmacological response for which the drug is administered in a significant number of subjects in need of such treatment. The “therapeutically effective amount” may vary according, for example, the physical condition of the patient, the age of the patient and the severity of the disease. It is emphasized that migraine headache is not well understood and the etiology of particular migraines vary, as does the response to particular drugs. Thus, reference to “specific pharmacological response for which the drug is administered in a significant number of subjects in need of such treatment” is a recognition that a “therapeutically effective amount”, administered to a particular subject in a particular instance will not always abort migraine onset or relieve an actual migraine headache, even though such dosage is deemed a “therapeutically effective amount” by those skilled in the art.

The composition of the present invention is selected so as to exert a therapeutic effect within 10-60 minutes post administration, preferably about 30 minutes post administration, more preferably about 20 minutes post administration.

The following examples are intended to be merely illustrative in nature and to be construed in a non-limitative fashion.

### Examples

Reference is now made to the following examples, which together with the above descriptions illustrate the invention in a non-limiting fashion.

### Example 1

### Formulation of 50 mg Tablets

Ingredients of tablets

| Ingredient | Function | mg/tablet | Quantity (g) |
|---|---|---|---|
| S-ethylisothiouronium | API | 50 | 300 |
| diethylphosphate | | | |
| Lactose USP | Filler/Binder | 50 | 300 |
| Anhydrous | | | |
| Colloidal Silicon | Glidant | 1 | 6 |
| Dioxide | | | |
| Lactose USP | Filler/Binder | 51 | 306 |
| Anhydrous | | | |
| Microcrystalline | Filler | 40 | 240 |
| Cellulose NF | | | |
| Crospovidone | Disintegrant | 4 | 24 |
| (Polyvinylpyrrolidone) | | | |
| Stearic acid | Lubricant | 4 | 24 |
| TOTAL | | 200 | 1,200 |

Coating Ingredients

| | % w/w | Quantity (g) |
|---|---|---|
| Pre-Coat | | |
| Purified Water USP | 84.80 | 10,975 |
| Opadry Clear | 15.00 | 2,000 |
| Vanillin | 0.20 | 25 |
| TOTAL | 100.00 | 13,000 |
| Film coat | | |
| Purified Water USP | 84.80 | 12,720 |
| Opadry White | 15.00 | 2,250 |
| Vanillin | 0.20 | 30 |
| TOTAL | 100.00 | 15,000 |
| Polishing Coat | | |
| Purified Water USP | 95.00 | 9,500 |
| Opadry Clear | 5.00 | 500 |
| TOTAL | 100.00 | 10,000 |

In some embodiments the tablet further comprises a final polishing coat comprising a composition comprising water and PEG 8000. This final polishing coat imparts sheen to the tablet.

Opadry white contains hypromellose (hydroxypropylmethylcellulose; HPMC), polyethylene glycol (PEG), polysorbate 80 and titanium oxide. Opadry clear contains hypromellose and polyethylene glycol.

### Formulation Steps

### Blending Procedure:

A. Collect into a container:
   1. S-ethylisothiouronium diethylphosphate 300 g
   2. Lactose USP anhydrous 300 g
   3. Colloidal Silicon Dioxide 6 g
B. Mix the ingredients together
C. Blend ingredients in blender.
D. Press mixture through #30 mesh screen and return mixture to blender.
E. To blender add:
   1. Lactose USP anhydrous 306 g
   2. Microcrystalline Cellulose NF 240 g
F. Blend for 5 minutes.
G. Add to mixture
   1. Crospovidone 24 g
   2. Stearic Acid 24 g
H. Mix and pass through #30 screen, and return mixture to blender.
I. Blend ingredients.

### Compression Step:

1. Transfer blend into hopper of compression machine
2. Collect compressed tablets.

### Coating Steps

### Pre-Coat

A. Add purified water USP (10,975 g) into stainless steel container
B. Under agitation, slowly add
   1. Opadry Clear 2,000 g
   2. Vanillin 25 g
C. Cover and keep for 1 hour.

### Film Coat

A. Add purified water USP (10,975 g) into stainless steel container
B. Under agitation, slowly add
   1. Opadry White 2,250 g
   2. Vanillin 30 g
C. Cover and keep for 1 hour.

### Polishing Coat

a. Add purified water USP (9,500 g) into stainless steel container
b. Under agitation, slowly add
1. Opadry Clear 500 g
c. Cover and keep for 1 hour.

### Pre-Coat

A. Add tablets into coating pan
B. Spray coat tablets at slow speed, 1 RPM with Pre-coating solution.

### Film Coat

A. Add tablets to coating pan
B. Spray coat tablets at slow speed, 1 RPM with Coating solution.

### Polishing Coat

A. Add tablets into coating pan
B. Spray coat tablets at slow speed, 1 RPM with Polishing solution.

### Example 2

### Tablet Dissolution

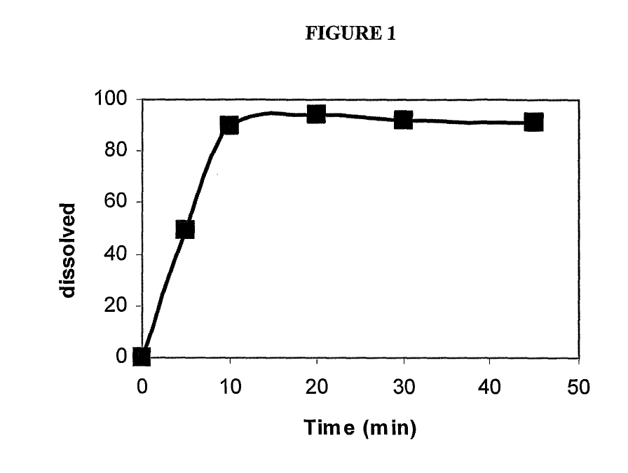

Dissolution was determined according to USP method 711 and as found to be about 94% in 20 minutes. FIG. 1 shows the dissolution profile of the composition. The Y axis shows % dissolution.

1.-27. (canceled)

28. A taste masked pharmaceutical composition for oral administration comprising as an active agent at least one S-alkylisothiouronium derivative and a taste masking effective amount of at least one polymeric coating.

29. The taste masked pharmaceutical composition according to claim 28, wherein the polymeric coating comprises a hydrophilic, water soluble polymer.

30. The taste masked pharmaceutical composition according to claim 29, wherein the hydrophilic, water soluble coating comprises a cellulose based compound.

31. The taste masked pharmaceutical composition according to claim 30, wherein the cellulose based compound is selected from the group consisting of methyl cellulose (MC), ethyl cellulose, hydroxypropylmethyl cellulose (HPMC), hydroxymethyl cellulose phthalate (HMCP), hydroxypropyl cellulose (HPC), cellulose acid phthalate and cellulose acetate phthalate.

32. The taste masked pharmaceutical composition according to claim 28, comprising a polymer coating comprising hydroxypropylmethyl cellulose and polyethylene glycol.

33. The taste masked pharmaceutical composition according to claim 28, further comprising a flavoring agent.

34. The taste masked pharmaceutical composition according to claim 33, wherein the flavoring agent is vanillin.

35. The taste masked pharmaceutical composition according to claim 28, comprising a polymeric precoat and a polymeric final coat.

36. The taste masked pharmaceutical composition according to claim 35, further comprising a polishing coat.

37. The taste masked pharmaceutical composition according to claim 36, wherein the polishing coat comprises a composition comprising a cellulose based material, a polyethylene glycol or a combination of a cellulose based material and a polyethylene glycol.

38. The taste masked pharmaceutical composition according to claim 28, wherein the S-alkylisothiouronium derivative is a compound having the general formula I: 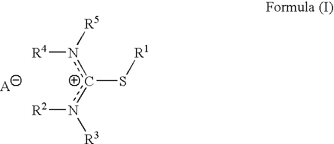
wherein,
R₁ is a linear or branched, saturated or unsaturated alkylene, comprising one to eight carbon atoms, optionally substituted with one or more substituent selected from the group consisting of halogen, primary, secondary or tertiary amine, primary, secondary or tertiary alcohol, or interrupted by one or more heteroatom selected from the group consisting of O, N, and S;
R₂, R₃, R₄ and R₅ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion derived from a phosphorous containing acid.

39. The taste masked composition according to claim 38, wherein the anion is selected from the group consisting of an anion derived from a phosphorus acid ester and an amide.

40. The taste masked composition according to claim 39, wherein the anion is derived from a mono or di-alkyl ester of a phosphorous containing acid.

41. The taste masked composition according to claim 38, wherein the compound is selected from the group consisting of:
S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

42. The taste masked composition according to claim 38, wherein the compound is S-ethylisothiouronium diethylphosphate.

43. The taste masked composition according to claim 28, wherein the composition is prepared as a tablet.

44. The taste masked composition according to claim 43, wherein the tablet comprises as an active agent between 10 and 200 mg of the S-alkylisothiouronium derivative.

45. The taste masked composition according to claim 28, wherein the composition is packaged and identified as having activity for treating or preventing one or more indications selected from the group consisting of a headache, migraine, nausea, emesis, low arterial blood pressure and hyperoxia.

46. The taste masked composition according to claim 28, comprising:
a. about 5% w/w to about 50% w/w of at least one S-alkylisothiouronium derivative according to formula I;
b. about 20% to about 90% of at least one filler-binder;
c. about 0.2% w/w to about 10% w/w of at least one lubricant, one glidant or a combination thereof;
d. about 0.2% to about 10% of at least one disintegrant; and
e. at least one polymer coating.

47. The taste masked composition according to claim 46, comprising:
a. about 5% w/w to about 50% w/w of S-alkyisothiuronium diethylphosphate;
b. about 20% to about 70% of at least one filler-binder selected from the group consisting of anhydrous lactose, microcrystalline cellulose, and a combination of lactose and microcrystalline cellulose;
c. about 0.2% w/w to about 10% w/w of at least one lubricant and/or glidant selected from the group consisting of colloidal silicon dioxide, stearic acid, talc, calcium stearate, magnesium stearate and sodium stearyl fumarate;
d. about 0.2% w/w to about 10% w/w of at least one disintegrant selected from crosslinked polyvinyl pyrrolidone, corn starch, potato starch, maize starch, and modified starches, croscarmellose sodium, crospovidone, sodium starch glycolate, and mixtures thereof; and
e. at least one polymer coating comprising hydroxypropylmethylcellulose and polyethylene glycol.

48. A method for the preparation of a taste masked tablet composition comprising as an active agent at least one S-alkylisothiouronium derivative, the method comprising the steps of:
a. blending a mixture of about 5% final w/w to about 50% final w/w of at least one S-alkylisothiouronium derivative according to formula I with 10% final w/w to about 40% final w/w filler binder and about 0.2% final w/w to about 10% final w/w of at least one lubricant and/or glidant;
b. adding to said blended mixture about 10% final w/w to about final 50% w/w filler binder, and blending;
c. adding to the blended mixture of step b) about from 0.2% final w/w to about 10% final w/w of at least one disintegrant; and about 0.2% final w/w to about 10% final w/w of at least one lubricant and or glidant and blending;
d. compressing the composition into tablets;
e. precoating the tablets with a polymer composition comprising hydroxypropylmethylcellulose and polyethylene glycol, and an optional flavoring agent;
f. coating the tablets with a polymer composition hydroxypropylmethylcellulose and polyethylene glycol, and an optional flavoring agent.

49. The method according to claim 48, further comprising the step of applying a polishing coat.
